(19) United States
(12) Patent Application Publication
(10) Pub. No.: US 2020/0102534 A1
(43) Pub. Date: Apr. 2, 2020

(54) AFFT2 CELL

(71) Applicant: BEIJING DCTY BIOTECH CO., LTD., Beijing (CN)

(72) Inventors: Shunchang Jiao, Beijing (CN); Rong Zhang, Beijing (CN); Zishan Zhou, Beijing (CN); Jiasen Xie, Beijing (CN); Xiaobin Chen, Beijing (CN); Gang Peng, Beijing (CN); Hongli Chen, Beijing (CN)

(21) Appl. No.: 16/503,802

(22) Filed: Jul. 5, 2019

(30) Foreign Application Priority Data
Sep. 30, 2018 (CN) 201811153265.X

(51) Int. Cl. C12N 5/0784; C07K 14/725; C12N 5/0783; C12N 15/86

(52) U.S. Cl. CPC C12N 5/064; C07K 14/7051; C12N 2740/15043; C12N 15/86; C12N 2810/80; C12N 5/0638

(57) ABSTRACT

The disclosure relates to an AFFT2 cell and a preparation method thereof. According to an embodiment, the cell is transformed with the TCR-T technology. The transformed T cells are blocked in vitro by an antibody drug of suppressive signaling molecules. According to another embodiment, a predicted antigen epitope is centered on a mutant amino acid site, the predicted antigen epitope extends 10 amino acids to each side of the mutant amino acid site, and the predicted antigen epitope serves as a potential antigen epitope. According to a further embodiment, TCR genes in a peripheral blood cell of a patient are knocked out by a CRISPR technology.

### CROSS REFERENCE TO RELATED APPLICATIONS

This application claims priority to Chinese application number 20181115326-5.X filed on Sep. 30, 2018, the disclosure of which is incorporated by reference herein in its entirety.

### FIELD OF THE DISCLOSURE

The disclosure relates generally to the field of biotechnologies. More specifically, the disclosure relates to an AFFT2 cell and a preparation method thereof.

### BACKGROUND

At present, in the specific immunotherapy of tumors, the existing LAK, DC, CIK, and DC-CIK cells and methods have basically proved to be ineffective, while cell technologies such as NK, CAR-NK, and TIL are not mature yet, CAR-T cells are also defective in the safety and treatment of solid tumors.

Some prior art references provide producing specific killing by DC-presenting T cells by transforming DC cells. Some laboratories are attempting to transfect the presenting T cells by using a virus as a vector to induce specific killing of the T cells. We have also directly stimulated Peripheral Blood Mononuclear Cell (PBMC) with mutant mixed polypeptide to induce the T cells. There are also laboratories that use the TCR-T technology to target presenting MAGE A3 antigens.

The foregoing treatment methods are not mature, especially the induction of DC cells in vitro and DC cell-bearing tumor antigen technologies are theoretically studied, but there are still many problems in the specific implementation process, lack of clear related molecules of signaling pathways for tumor cell development and progression acts as inducing antigens, because of unknown tumor antigens and barriers of tumor microenvironmental immunosuppression, which makes it difficult to implement specific cell-targeted immunotherapy. In addition, although some antigen-pulsed methods in vitro have been carried out, co-culture in vitro and amplification in vitro have not been carried out, so that relatively weak specific cells directly face the complex tumor immune microenvironment, and therefore, it is difficult to achieve the desired effect. Some may also be presented in vitro and co-cultured, but the target is single (MAGE-3), which only works for individual cancers such as the non-small cell lung cancer. Although it is also attempted to transfect presentation by using a lentivirus as the vector, the safety and convenience are inferior to the polypeptide. Moreover, direct stimulation with mixed polypeptide is simple but the efficiency is low. Secondary stimulation with specific polypeptide is less direct than transduction of tumor-specific TCR. The existing TCR-T lacks a precise TCR covering more types of tumors in the solutions for treating hematological and solid tumors.

None of the foregoing solutions considers the self-protection technology of T cells. As a result, a small number of specific T cells directly face the powerful tumor immune microenvironment.

### SUMMARY

The following presents a simplified summary of the invention in order to provide a basic understanding of some aspects of the invention. This summary is not an extensive overview of the invention. It is not intended to identify critical elements or to delineate the scope of the invention. Its sole purpose is to present some concepts of the invention in a simplified form as a prelude to the more detailed description that is presented elsewhere.

In some embodiments, a preparation method of an AFFT2 cell includes the following steps. 1. Drawing peripheral blood of a patient to perform ctDNA exon sequencing or performing whole exon sequencing with tumor tissues. 2. Screening a mutation site to perform antigenic epitope prediction, and synthesizing a mutant polypeptide. 3. Using the peripheral blood to prepare an immortalized DC, and loading the mutant polypeptide, and co-incubating with a PBMC to obtain an AFF cell. 4. Using the mutant polypeptide as an antigen to stimulate the AFF cell, and screening to obtain a precise polypeptide. 5. Loading the immortalized DC cell with the precise polypeptide, and co-incubating with the PBMC to prepare an AFF′ cell. 6. Using the precise polypeptide as an antigen to stimulate the AFF′ cell, and screening to obtain a specific T cell capable of recognizing the precise polypeptide, and sequencing to obtain a high-frequency TCR sequence of the specific cells. 7. Isolating CD8+T cells from the PBMC, knocking out the original TCR, and performing high-frequency TCR expression to construct a TCR-T cell. 8. Blocking the TCR-T cell with a monoclonal antibody drug of suppressive signaling molecules to prepare the AFFT2 cell.

In other embodiments, an AFFT2 cell is prepared by the following steps.

1. Whole exon sequencing. Human peripheral blood is used for ctDNA sequencing or whole exon sequencing is performed with tumor tissues, the sequencing result is compared with a genome of normal cells, and mutation sites are screened out. the peripheral blood can also be a commercially available engineered cell line, such as H1299, H226, H358, H1563, H2228, A549, Renca, LLC mouse Lewis lung cancer cells, CRL-6323 B16F1, CRL-2539 4T1, U14 mouse cervical cancer cells, BV-2 mouse microglioma cells, or G422 mouse glioma cells, which is subjected to whole exon sequencing.

2. Antigen epitope prediction. The antigen epitope prediction is centered on a mutant amino acid site, extends 10 amino acids to each side, and the polypeptide segment having 21 amino acids is used as “a potential antigen epitope”. IC50 of the potential antigen epitope is analyzed by using prediction software, and if the IC50<1,000 nM, the potential antigen epitope is determined as “an antigen epitope”.

3. Loading of mutant polypeptide with immortalized DC. Dendritic cells in the peripheral blood are infected with TAX-GFP lentivirus, and the ideal clone is selected as the immortalized DC. “The antigen epitope” is synthesized into the mutant polypeptide to be loaded on the immortalized DC.

4. Co-incubation of the DC loaded with mutant polypeptides with PBMC. The DC loaded with the mutant polypeptides is co-cultured with the PBMC to obtain AFF cells.

5. Screening for precise polypeptide. The AFF cells are collected, and each of the synthesized mutant polypeptides is used to stimulate the AFF cells alone, and the precise polypeptide is screened by examining the secretion of IFN-γ.

6. Preparation of AFF′ cells with screened precise polypeptide. The mutant polypeptides are replaced with the precise polypeptide to repeat steps 3-(2) and 4 to prepare the precise polypeptide AFF′ cells.

7. Determination of specific cell high-frequency TCR and construction of an expression vector. The AFF′ cells are stimulated with the precise polypeptide. CD8, CD137, and IFN-γ staining is performed on the stimulated cells. CD8+CD137+ or CD8+IFN-γ+T cells are selected. The genome is extracted, and sequencing analysis is performed on the TCR. A high-frequency TCR sequence is determined based on the TCR distribution frequency. A primer is designed according to the high-frequency TCR sequence, and is amplified to obtain the TCR gene. A TCR gene expression vector is constructed, and the virus is packaged.

8. Construction of a CRISPR vector that knocks out the original TCR, and virus packaging.

9. Construction of AFFT cells. The virus obtained in step 8 is used to infect CD8+T cells for knocking out the original TCR, and then transferring the lentivirus of the TCR expression vector constructed in step 7.

10. The cells obtained in step 9 are blocked by the monoclonal antibody drug of suppressive signaling molecules, to prepare the AFFT2 cells. The suppressive signaling molecules can be one or more of PD-1, Tim-3, LAG3, CTLA-4, BTLA, VISTA, TIGIT, CD160, and 2B4 (CD244).

### BRIEF DESCRIPTION OF THE DRAWINGS

FIG. 1 shows microscopic observation of DC morphology.

FIG. 2 shows the efficiency of loading polypeptides with DC.

FIG. 3 shows screening of precise polypeptide.

FIG. 4 shows flow cytometry detection of specific T cell proportion.

FIG. 5 shows TCR distribution frequency.

FIG. 6 shows detection of knockout efficiency of original TCR.

FIG. 7 shows the expression efficiency of specific TCR.

FIG. 8 shows the proportion of cells capable of recognizing polypeptide antigen in the AFFT cells in the flow cytometry detection.

FIG. 9 shows the in vitro blocking efficiency of the monoclonal antibody drug.

FIG. 10 shows the killing ability of the effector cell to target cells.

FIG. 11 shows ELISA detection of the release of cytokine IFN-γ.

FIG. 12 shows the improvement of cells to the survival of tumor-bearing mice.

### DETAILED DESCRIPTION

The following describes multiple exemplary embodiments of the disclosure with reference to the accompanying drawings in the embodiments of the disclosure. The described embodiments are merely a part rather than all of the embodiments of the present invention. All other embodiments obtained by a person of ordinary skill in the art based on the embodiments of the present invention without creative efforts shall fall within the protection scope of the present invention.

Regarding the term “AFFT2” in the disclosure, “A” represents immortalized dendritic cell (“DC”) technology, “FF” represents mixed polypeptide stimulation technology, “T” represents TCR-T technology, “2” represents antibody in vitro blocking protection technology. AFFT2 cells are products obtained by a combination of the above techniques.

In an embodiment, a lung cancer patient is taken as an example to provide an antigen specific AFFT2 cell and a preparation method thereof. The preparation method of a AFFT2 cell includes the following steps.

1. Whole Exon Sequencing

The peripheral blood of the lung cancer patient is drawn for ctDNA sequencing and HLA typing detection. The sequencing information is analyzed with software, the ctDNA sequencing result is compared with a genome of normal cells, and mutation sites are screened out.

2. Antigen Epitope Prediction

The antigen epitope prediction is centered on a mutant amino acid site, extends 10 amino acids to each side, and the polypeptide segment having 21 amino acids is used as “a potential antigen epitope”. IC50 of the potential antigen epitope is analyzed with prediction software (recommended software: NetMHCpan 3.0, PickPocket, and Artificial Neural Networks (ANN)), and if IC50<1,000 nM, this potential antigen epitope is considered to be an “antigen epitope”.

3. Synthesis of Polypeptide

A technology service company is entrusted to synthesize the “mutant epitope” to synthesize a mutant polypeptide.

4. Immortalization of DC

100 ml of peripheral blood of the patient is drawn. PBMC is isolated by Ficoll density gradient centrifugation. The dendritic cells are isolated using a Miltenyi dendritic cell isolation kit and re-suspended in a culture medium. The isolated dendritic cells are infected by TAX-GFP lentivirus, statically cultured at 37° C. in an incubator and observed. The cloned and grown cells are selected to be cloned in a 96-well plate for culture, respectively. Monoclonal phenotypic analysis. The ideal clone is used as the immortalized DC.

5. Loading of Mutant Polypeptide with the Immortalized DC

Preparation of a polypeptide solution: the mutant polypeptide synthesized in step 3 is adopted for preparing, and the final concentration of each polypeptide is 10-100 μg/mL, preferably 50 μg/mL, for standby. The obtained immortalized DC is centrifugally collected, re-suspended in the prepared polypeptide solution, and placed in a cell culture plate for polypeptide loading. The cell culture plate is pulsed in 5% CO₂ at 37° C. for 1-4 h, preferably 4 h, for standby.

6. Co-Incubation of PBMC and DCs Loaded with Mutant Polypeptides

A pre-coating plate of stimulating factor OKM-25, i.e., 40 μL of OKM-25+4 mL of PBS, 2 mL/dish (4.5 cm2) is placed at room temperature for 4 h, 4° C. for standby. The DC loaded with the mutant polypeptide and the PBMC are mixed at a ratio of 1:50 to 1:500, preferably 1:100, and transferred to a cell culture plate or a culture flask pre-coated with OMK-25. The mixture is mixed well, cultured in 5% CO₂ at 37° C., and recorded as day 0. The condition of the co-cultured cells is observed. The co-cultured cells are transferred to a large culture flask on the 5th day according to the cell density, and 20 mL of fresh culture medium OKM-100+12% FBS is supplemented in a 75 cm2 culture flask. 20 mL of fresh OKM-100+12% FBS is added on the 7th day of co-culture. The culture medium is OKM-200+5% FBS, and the co-cultured cells are transferred from the 75 cm2 flask to the 175 cm2 large flask on the 10th day of co-culture. The transfer method is: cells are dispersed with 25 mL culture medium OKM-200+5% FBS, and then transferred to a large flask and repeated twice, and makes up to 200 mL with the culture medium OKM-200+5% FBS. 8) the cells of the AFF scheme are obtained when cultured for 14-21 days.

7. Direct Stimulation of T Cells with the Polypeptide as an Antigen to Screen the Precise Polypeptide

The obtained cells of the AFF scheme are centrifugally collected, and centrifuged at 1,500 rpm for 5 min to collect T cells. 10 mL of PBS is added to re-suspend the cells, and counted, centrifuged at 1,500 rpm for 5 min, and the T cells are collected and re-suspended in 1640+10% FBS+200 U/mL IL2, and the cell density is adjusted to 1×10⁶ cells/mL. The T cells are distributed into a 96-well flat-bottom plate at 200 μL/well with a micropipette, and the number of cells is 2×105 cells. Then 10 μL of 1 mg/mL mutant polypeptide synthesize in step 3 is added, and a final concentration is 50 μg/mL, each polypeptide is provided with three holes. A positive control: T cell +100 ng/mL OKT3, and a negative control: 1640+10% FBS+200 U/mL IL2. Two T cell controls are used as background release detection, T cells are added at earliest time and latest time. The difference in the release of two backgrounds as a systematic error. The cells are stimulated in 5% CO₂ at 37° C. for 24 h, and centrifuged at 1,500 rpm for 10 min, and 140 μL of supernatant is transferred to a new 96-well plate. The 96-well plate is centrifuged at 1,500 rpm for 10 min, and a sample is taken for ELISA detection (or the sample is stored at −80° C.).

8. Evaluation Criteria of Precise Polypeptide

If the positive control and the negative control are normal, the data is reliable. T cells as baseline when polypeptide serving as antigen. Each group of experiments consists of two baselines: a high baseline and a low baseline. The high baseline has a high detection value, the low baseline has a low detection value, and the difference between the two baselines is the systematic error, and during data analysis, the detection value >the low baseline, >the high baseline and >the high baseline+the systematic error are labeled separately. The detection value >the high baseline+the systematic error is a valid precise polypeptide.

9. Preparation of AFF′ Cells with the Screened Precise Polypeptide

The precise polypeptide AFF′ cells are prepared by methods 4, 5, and 6.

10. Culture and Isolation of Mutant Antigen Specific Killing T Cells

The AFF′ cells obtained in step 9 are directly stimulated by the screened precise polypeptide for 12-72 h for standby. The stimulated T cells are stained with CD8, CD137, IFN-γ, and are sorted by a flow cytometer, and CD8+CD137+ or CD8+IFN-γ+ cells are selected.

11. TCR Frequency Detection of CD8+T Cells and Cloning of High-Frequency TCR

The cells are obtained by sorting, and the genome is immediately extracted. The genome is subjected to TCR sequencing analysis, and the high-frequency TCR sequence is determined according to the TCR distribution frequency. The mRNA of the PBMC is extracted, reversely transcribed into cDNA, and primers are designed according to the high-frequency TCR sequence, and amplified to obtain the TCR gene. A TCR gene expression vector is constructed, and the virus is packaged.

12. Construction of a CRISPR Vector that Knocks Out the Original TCR

A CDS region of mRNA of the TCR gene is found on Pubmed, and a conserved region of the TCR is analyzed, and the conserved region is subjected to knockout target prediction. The construction of the TCR knockout vector and virus packaging are completed by the following steps. Forward primers and reverse primers required for synthesizing sgRNA are designed, and the forward primers and the reverse primers are mixed at 1:1, and then treated at 95° C. for 5-60 min, and then slowly cooled to form a DNA sequence of the sgRNA. The CRISPR lentiviral vector is double-digested and ligated with the double-stranded DNA corresponding to the sgRNA, and transferred into the cloned competent cells, the mono clones are picked for sequencing after 12 h, and the clones of correct sequencing are retained. A CRISPR lentiviral vector carrying the DNA sequence corresponding to the sgRNA is extracted for virus packaging.

13. Construction of AFFT Cells

PBMC is thawed, and CD8+T cells are sorted with magnetic beads. The virus obtained in step 12 is used to infect the CD8+T cells for knocking out the original TCR. The infected CD8+T cells are cultured in the culture medium for 0-5 days, preferably 3 days, and then transfected by the lentivirus constructed in step 11. The infected CD8+T cells are re-suspended in OKM100+12% FBS, and placed on a pre-coating plate of the stimulating factor OKM-25, recorded as day 0. The cell conditions and the cell density are observed. The co-cultured cells are transferred to a large culture flask at the 5th day, and fresh culture medium OKM-100+12% FBS is supplemented. The cells are transferred from a 75 cm2 flask to a 175 cm2 large flask, and the culture medium is OKM-200+5% FBS. The TCR-T can be harvested when cultured for 14-21 days to obtain the AFFT cells.

14. Blocking of Immunosuppressive Signaling

The immunosuppressive signaling molecule is PD-1. The cultured TCR-T cells are collected by centrifuging at 1,000 rpm for 5 min. The cells are washed with PBS once and centrifuged at 1,000 rpm for 5 min, the TCR-T cells are re-suspended with OKM-200+5% FBS, and adjusted to 1×107/mL. The monoclonal antibody drug Keytruda of the suppressive signaling molecules is added at a final concentration of 50-500 μg/mL, preferably 150 μg/mL, and blocked at 0-37° C. for 1-4 h, preferably at 37° C. for 1 h to obtain the AFFT2 cells.

15. Construction of Specific Antigen Expression Target Cells and Tumor Model Survival Experiments

A lentiviral vector that can express the screened precise polypeptide (specific antigen) is constructed. The specific antigen-expressing lentiviral vector is packaged into lentiviral particles, appropriate HLA-matched tumor cells are infected, specific antigens are stably over-expressed, and the expression level and expression intensity are flow-detected. NGS mice are inoculated with the tumor cell line stably overexpressing specific antigen peptides to serve as ectopic tumor-bearing animal models. 5×105 tumor cells expressing the specific antigens are suspended in 100 μl of physiological saline, subcutaneously injected to the right flank of 30 NSG mice, and the mice are numbered. The cells are infused in groups when the tumor grows to about 100-120 mm3. According to the tumor volume, the animal models are randomly divided into three groups, each group of 5-6 mice, one group is given placebo saline, one group is given 1×107 of T cells (control group) without any genetic manipulation, and one group is given 1×107 of AFFT2 cells, the second injection is performed 7 days after the first injection of cells, and the cells are injected for the third time after 7 days, observation is made for 60 days continuously, the survival data is counted, and the survival curve is plotted.

### Experimental Results

1. Mutation Site and Antigen Epitope Prediction

Table 1 shows a result of mutation sites detected by sequencing and antigen epitope prediction, and the mutant amino acid is underlined.

TABLE 1
Antigen epitope prediction

| Polypeptide | Antigen | Affinity to |
|---|---|---|
| No. | Epitope | HLA (nM) |
| 1 | HSFQAIEL | 121.34 |
| 2 | WLIHSFQAIEL | 40.68 |
| 3 | LLLFIKHTY | 26.83 |
| 4 | GAHRDAAKI | 13.89 |
| 5 | GGYGYISAC | 15.94 |
| 6 | FIVNTLNAGL | 53.61 |
| 7 | ASYGAHRDA | 3.7 |
| 8 | VLDMLCLGI | 65.8 |
| 9 | FILMELEVL | 14.51 |
| 10 | EPATKLPPL | 10.71 |

2. Morphologic Observation of Immortalized DC

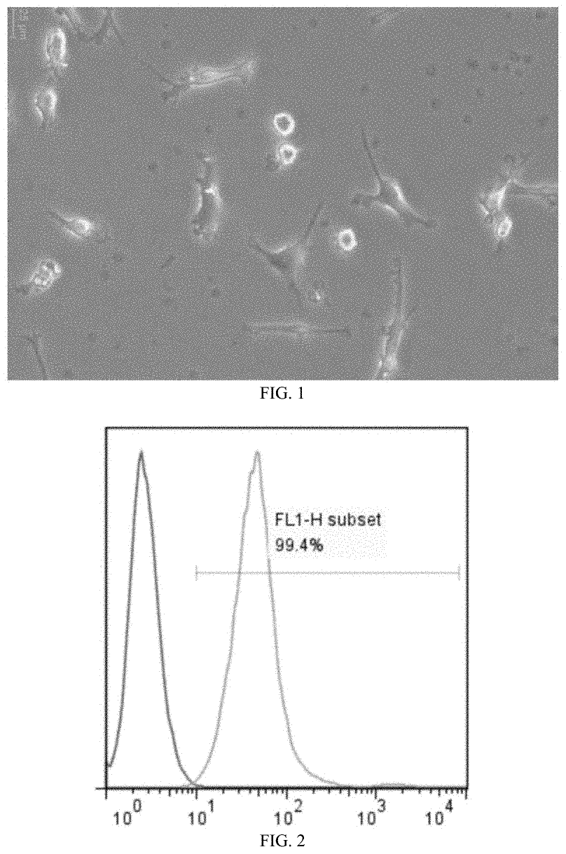

The morphology of the mature induced DC is observed with a microscope first, and distinct dendritic cells can be observed (FIG. 1).

3. Detection of DC Antigen Loading Efficiency

The predicted mutant antigens are synthesized according to Table 1, and biotins are labeled. After the antigen is loaded with DC, the distribution of biotins on the cell surface is detected by PE-labeled affinity streptomycin to detect the efficiency of DC presenting the polypeptide antigen. The results are as shown in FIG. 2: dark color (left) is the detection result without the labeled polypeptide, and light color (right) is the detection result of the loaded biotin polypeptide. The results show that the loading efficiency of DC is 99.4%.

4. Screening for Precise Polypeptides with AFF Cells

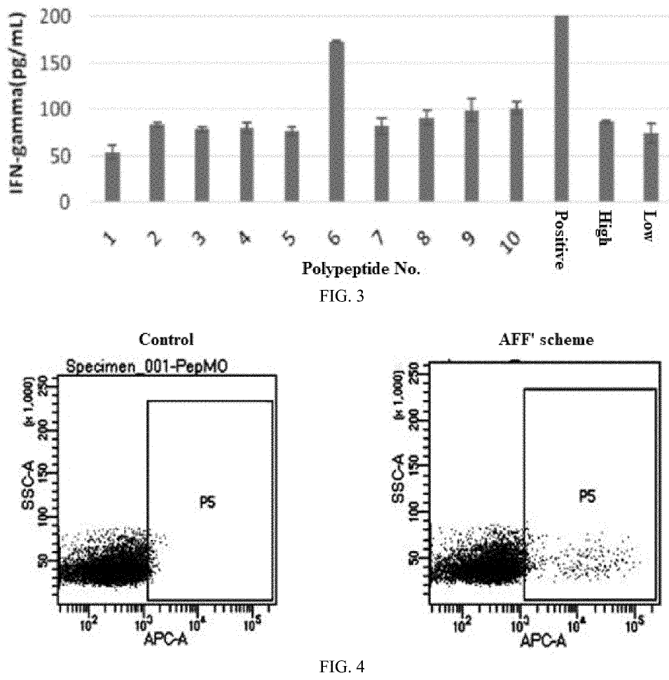

the cultured T cells are stimulated with 10 polypeptides, and the effective polypeptides are detected by detecting the secretion of IFN-γ. The results are as shown in FIG. 3: the release amount of IFN-γ by No. 6 polypeptide >high baseline+system error, which is an effective precise polypeptide.

5. Identification and Sorting of Specific T Cells for Precise Polypeptides

The cells of the AFF′ scheme are stimulated by the selected No. 6 polypeptide, and the ratio of specific T cells for the precise polypeptide is detected in a flow cytometry mode. The results are as shown in FIG. 4. The black box (P5) is a specific T cell: the cells of the AFF′ scheme, the proportion of cells releasing IFN-γ caused by polypeptide No. 6 is significantly higher than that of cells without stimulation (control), indicating that the AFF′ scheme can obtain specific T cells for precise polypeptide. Sorting of the CD8+IFN-γ+ cells (black box) is performed by the flow cytometer.

6. Identification and Cloning of High-Frequency TCR

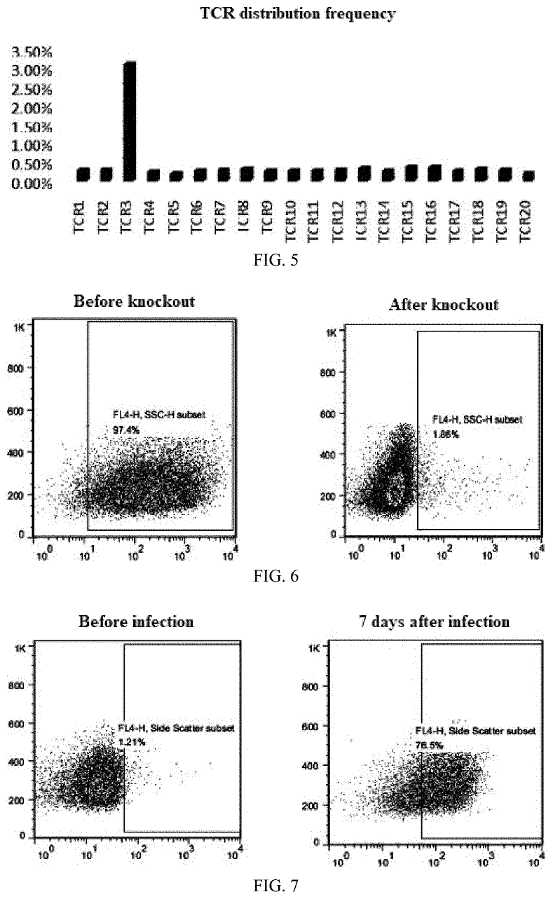

Genome extraction on the sorted cells, sequencing of TCR, and the distribution of TCR are as shown in FIG. 5 (the top 20 of the high-frequency distribution), and the TCR3 distribution frequency is high, indicating that the TCR is closely related to the mutant antigen, and the TCR is amplified according to the TCR sequence, to construct a lentiviral expression vector.

TABLE 2
Sequence of TCR β chain CDR3

| No. | DNA sequence 5′-3′ of TCR β chain CDR | CDR3 amino acid sequence |
|---|---|---|
| TCR1 | TGTGCCAGCAGTTCCCGACTAGCGGGGGGGACAGATACGCAGTATTTT | CASSSRLAGGTDTQYF |
| TCR2 | TGTGCCACCAGCAGGGAGGGAGGGGAGACCCAGTACTTC | CATSREGGETQYT |
| TCR 3 | TGTGCCACCAGCAGAGATTGGCTATCCAACGGGAACACTGAAGCTTTCTTT | CATSRDWLSNGNTEAFP |
| TCR 4 | TGTGCCAGCAGTTACTCGCTCGGAATCTACGAGCAGTACTTC | CASSYSLGIYEQYF |
| TCR 5 | TGTGCCAGCAGTGATTTCGGACCATCGTGGGGCAATGAGCAGTTCTTC | CASSDFGPSWGNEQFF |
| TCR 6 | TGTGCCAGCAGCTTAGAGGCAGTGAACACTGAAGCTTTCTTT | CASSLEAVNTEAFF |
| TCR 7 | TGTGCCAGCAGCCCCGTTGGCGGCCAAGGAACCCTCCACTTT | CASSPVGGQGTLHF |
| TCR 8 | TGTGCCAGCAGCTTAGGGACACTATATGGCTACACCTTC | CASSLGTLYGYTF |
| TCR 9 | TGCGCCAGCAGTGAGTCTGGGACAGGGTTCTCCTACGAGCAGTACTTC | CASSESGTGFSYEQYF |
| TCR 10 | TGTGCCAGTAGTGCGGCAGGGGGGCCCTACGAGCAGTACTTC | CASSAAGGPYEQYF |
| TCR 11 | TGTGCCATCAGTGAGTCGGACAGTCCCATCGACACTGAAGCTTTCTTT | CAISESDSPIDTEAFF |
| TCR 12 | TGCGCCAGCAGCTTGGCCGAGGGAGATGGCTACACCTTC | CASSLAEGDGYTF |
| TCR 13 | TGTGCCACCAGCAGAGTGGCCGGGAGACCCAGTACTTC | CATSRVAGETQYF |
| TCR 14 | TGTGCCACCAGTGATGGGGCAGGGGTCGAGCAGTTCTTC | CATSDGAGVEQFF |
| TCR 15 | TGTGCCAGCAGTTTGGCGGAGATCAATCAGCCCCAGCATTTT | CASSLAEINQPQHF |
| TCR 16 | TGTGCCAGCAGCTCTTCAAGCGGGACCGGGGCTTTCTTT | CASSSSSGTGAFF |
| TCR 17 | TGTGCCAGCAGCTCCCTTGGGGTCACTGAAGCTTTCTTT | CASSSLGVTEAFF |
| TCR 18 | TGTGCCAGCAGCCCACCGGGCTTGAATGAAAAACTGTTTTTT | CASSPPGLNEKLFF |
| TCR 19 | TGTGCCAGCAGTGAAGAGGGCAGCCCCCGGGGATACGAGCAGTACTTC | CASSEEGSPRGYEQYF |
| TCR 20 | TGCGCCAGCAGTCGGACAGGGGAGAATTCACCCCTCCACTTT | CASSRTGENSPLHF |

Known TCR-α:

Amino acid sequence:
MMKSLRVLLV ILWLQLSWVW SQQKEVEQNS GPLSVPEGAI
ASLNCTYSDR GSQSFFWYRQ YSGKSPELIM FIYSNGDKED
GRFTAQLNKA SQYVSLLIRD SQPSDSATYL CAVNFGGGKL
IFGQGTELSV KPN
Nucleic acid sequence:
ATGATGAAAT CCTTGAGAGT TTTACTAGTG ATCCTGTGGC
TTCAGTTGAG CTGGGTTTGG AGCCAACAGA AGGAGGTGGA
GCAGAATTCT GGACCCCTCA GTGTTCCAGA GGGAGCCATT
GCCTCTCTCA ACTGCACTTA CAGTGACCGA GGTTCCCAGT
CCTTCTTCTG GTACAGACAA TATTCTGGGA AAAGCCCTGA
GTTGATAATG TTCATATACT CCAATGGTGA CAAAGAAGAT
GGAAGGTTTA CAGCACAGCT CAATAAAGCC AGCCAGTATG
TTTCTCTGCT CATCAGAGAC TCCCAGCCCA GTGATTCAGC
CACCTACCTC TGTCCCGTGA ACTTCGGAGG AGGAAAGCTT
ATCTTCGGAC AGGGAACGGA GTTATCTGTG AAACCCAAT

Known TCR-β:

Amino acid:
MRIRLLCCVA FSLLWAGPVI AGITQAPTSQ ILAAGRRMTL
RCTQDMRHNA MYWYRQDLGL GLRLIHYSNT AGTTGKGEVP
DGYSVSRANT DDFPLTLASA VPSQTSVYFC ASSLSFGTEA
FFGQGTRLTV V (The transverse line is the CDR3
sequence, which is a sequence to be
substituted)
Substituted TCR-β:
MRIRLLCCVA FSLLWAGPVI AGITQAPTSQ ILAAGRRMTL
RCTQDMRHNA MYWYRQDLGL GLRLIHYSNT AGTTGKGEVP
DGYSVSRANT DDFPLTLASA VPSQTSVYFC
ATSRDWLSNGNTEAFFGQGTRLTVV (The transverse line
is the substituted CDR3 sequence)

7. Detection of Knockout Efficiency of Original TCR

The CRISPR technology is used to knock out the original TCR on the CD8+T cells. The result is as shown in FIG. 6. It can effectively reduce the expression of the original TCR. At this time, the transfection of the expression-specific TCR lentivirus can be performed.

8. Detection of Specific TCR Expression

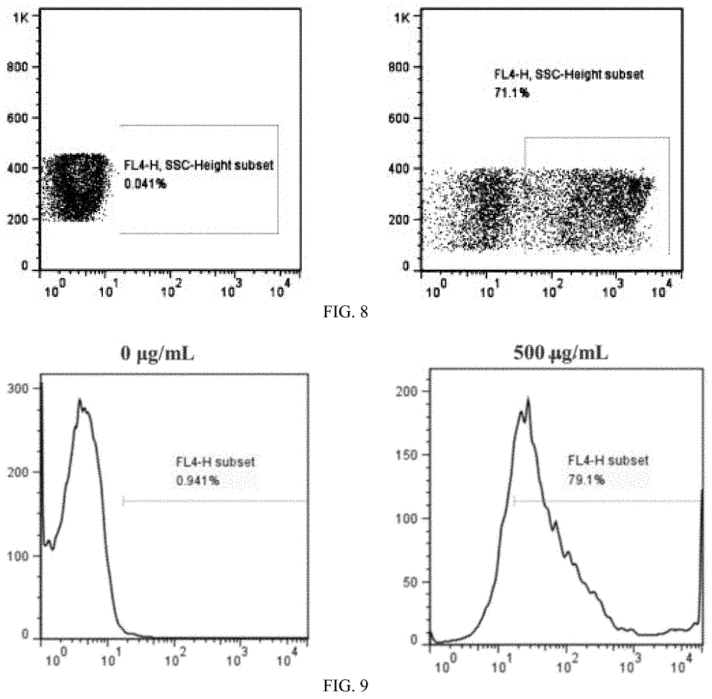

The foregoing cells are transfected with lentiviruses that package specific TCR. The expression efficiency of TCR is detected in a flow cytometry mode on the 7th day. The results are as shown in FIG. 7. The constructed TCR can be expressed normally, and the ratio of TCR+ cells is 76.5%. The proportion of specific cells recognizing the polypeptide antigen is 71.1% (FIG. 8).

9. Blocking Effect of Suppressive Signaling of the AFFT2 Cells

500 μg/mL of fluorescently labeled monoclonal antibody drug Keytruda is added to the PBS buffer system. As shown in FIG. 9, 79.1% of the cells are effectively blocked.

10. The Killing Ability of the AFFT2 Cells to Target Cells

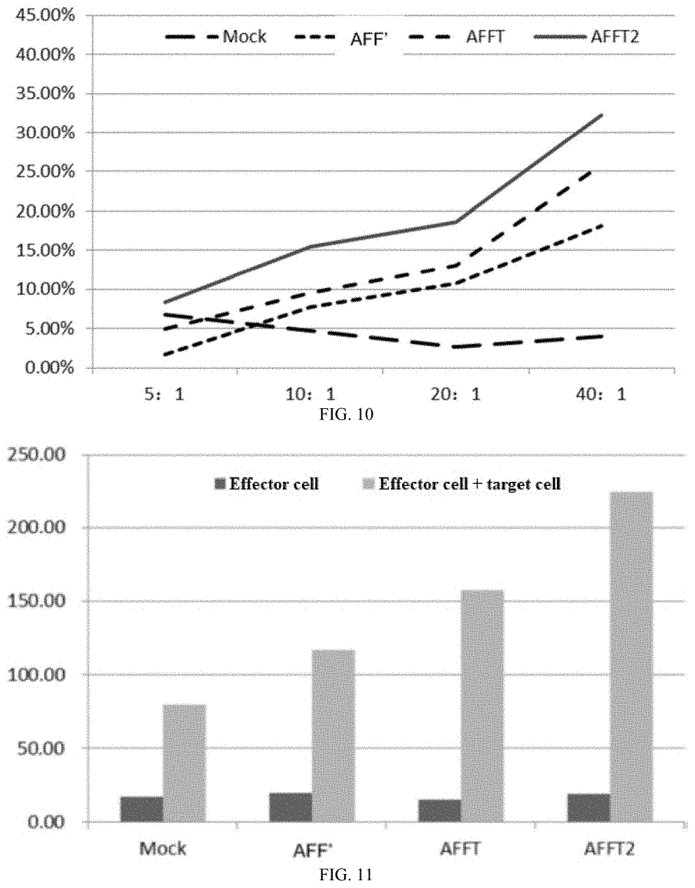

The killing efficiencies of the AFF′ cells, AFFT cells and AFFT2 cells to the target cells derived from the mutant antigen epitope are detected, respectively, and the untreated cells are used as a control (Mock). The results are as shown in FIG. 10. Compared with the control group, the AFF′ cells, AFFT cells and AFFT2 cells have certain killing effects on the target cells, and are significantly different from Mock group at 10:1, 20:1 and 40:1 (effector cells: target cells). After the suppressive signaling molecules are blocked, AFFT2 cells >AFFT cells >AFF′ cells in the killing efficiency against the tumors, indicating that the T cells expressing the specific TCR, together with the blocking of the suppressive target, can effectively improve the killing efficiency of the tumor cells.

11. Detection of Cytokines Released by the AFFT2 Cell

During co-culture of the tumor cells and the effector cells, mutant antigens on the tumor cells can be recognized due to the effector cells. Therefore, a series of cytokines are produced. IFN-γ is one of the most important cytokines against tumors. FIG. 11 shows the detection of the released IFN-γ when the cells cultured in different modes are co-cultured with tumor cells. The result shows that compared with IFN-γ produced by the effector cells, the AFF′ scheme cells, the AFFT scheme cells, and the AFFT2 cells can produce a large amount of IFN-γ after being co-cultured with tumor cells, particularly the AFFT and AFFT2 cells, since the specific TCR (AFFT) is expressed, the suppressive signaling is blocked (AFFT2), and the effector cells can release more IFN-γ. This result and the killing experiment results indicate that the T cells expressing the specific TCR, together with knockout of a suppressive target, can enhance the anti-tumor ability more effectively.

12. Construction of Specific Antigen-Expressing Target Cells and Tumor Model Survival Experiments

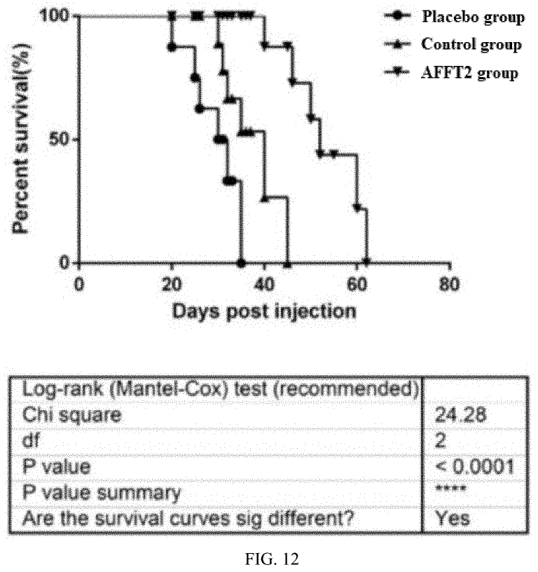

A specific antigen-expressing tumor target cell line is successfully constructed, and a tumor-bearing animal model is established. The result (FIG. 12) shows that the AFFT2 cell has a significant effect on the survival improvement of the tumor-bearing mice.

Various embodiments of the disclosure may have one or more of the following effects. The AFFT2 cell provided by the disclosure uses a tumor antigen as a mutant antigen, which is different from other tissues. The tumor antigen may have a strong target specificity, may not be easy to cause off-target effects, and may have high safety. The proportion of the specific cells obtained by the disclosure may be high. The distribution of the specific cells capable of recognizing tumor antigens in PBMC may be 0.5% or less. The proportion of the cells transformed by the AFFT2 scheme and the specific T cells (TCR+) recognizing the tumor antigens may be 70% or more. The AFFT2 cell obtained by the disclosure may block the immunosuppressive targets such as PD1, CTLA4, TIM3, and LAG3 with the monoclonal antibody drug. The killing ability against tumors may not be limited, and the killing efficiency be higher. An AFFT2 cell may be transformed with the TCR-T technology, and the transformed T cells may be blocked in vitro by an antibody drug of suppressive signaling molecules, which may enhance the anti-tumor ability of the T cells.

Many different arrangements of the various components depicted, as well as components not shown, are possible without departing from the spirit and scope of the present disclosure. Embodiments of the present disclosure have been described with the intent to be illustrative rather than restrictive. Alternative embodiments will become apparent to those skilled in the art that do not depart from its scope. A skilled artisan may develop alternative means of implementing the aforementioned improvements without departing from the scope of the present disclosure.

It will be understood that certain features and subcombinations are of utility and may be employed without reference to other features and subcombinations and are contemplated within the scope of the claims. Unless indicated otherwise, not all steps listed in the various figures need be carried out in the specific order described.

The disclosure claimed is:

1. An AFFT2 cell, prepared by a method comprising the steps of:
1) drawing peripheral blood of a patient to perform ctDNA exon sequencing or performing whole exon sequencing with tumor tissues;
2) screening a mutation site to perform antigenic epitope prediction, and synthesizing a mutant polypeptide;
3) using the peripheral blood to prepare an immortalized dendritic cell, loading the mutant polypeptide, and co-incubating with a PBMC to obtain an AFF cell;
4) using the mutant polypeptide as an antigen to stimulate the AFF cell, and screening to obtain a precise polypeptide;
5) loading the immortalized dendritic cell with the precise polypeptide, and co-incubating with the PBMC to prepare an AFF′ cell;
6) using the precise polypeptide as an antigen to stimulate the AFF′ cell, screening to obtain a specific T cell capable of recognizing the precise polypeptide, and sequencing to obtain a high-frequency TCR sequence of the specific cells;
7) isolating CD8+T cells from the PBMC, knocking out the original TCR, and performing high-frequency TCR expression to construct a TCR-T cell; and
8) blocking the TCR-T cell with a monoclonal antibody drug of suppressive signaling molecules to prepare the AFFT2 cell.

2. The AFFT2 cell according to claim 1, wherein the peripheral blood of the patient is a commercially available engineered cell line.

3. The AFFT2 cell according to claim 1, wherein:
a predicted antigen epitope is centered on a mutant amino acid site;
the predicted antigen epitope extends 10 amino acids to each side of the mutant amino acid site; and
the predicted antigen epitope serves as a potential antigen epitope.

4. The AFFT2 cell according to claim 1, wherein a method of knocking out TCR genes in a peripheral blood cell of the patient is a CRISPR technology.

5. The AFFT2 cell according to claim 1, wherein the suppressive signaling molecule comprises one or more of PD-1, Tim-3, LAG3, CTLA-4, BTLA, VISTA, CD160, 2B4(CD244), and TIGIT.


SEQUENCE LISTING (54 sequences)

SEQ ID NO: 1 - protein, 8 aa, artificial sequence
  Antigen epitope prediction 1
HSFQAIEL

SEQ ID NO: 2 - protein, 11 aa, artificial sequence
  Antigen epitope prediction 2
WLIHSFQAIEL

SEQ ID NO: 3 - protein, 9 aa, artificial sequence
  Antigen epitope prediction
LLLFIKHTY

SEQ ID NO: 4 - protein, 9 aa, artificial sequence
  Antigen epitope prediction 4
GAHRDAAKI

SEQ ID NO: 5 - protein, 9 aa, artificial sequence
  Antigen epitope prediction 5
GGYGVISAC

SEQ ID NO: 6 - protein, 10 aa, artificial sequence
  Antigen epitope prediction 6
FIVNTLNAGL

SEQ ID NO: 7 - protein, 9 aa, artificial sequence
  Antigen epitope prediction 7
ASYGAHRDA

SEQ ID NO: 8 - protein, 9 aa, artificial sequence
  Antigen epitope prediction 8
VLDMLCLGI

SEQ ID NO: 9 - protein, 9 aa, artificial sequence
  Antigen epitope prediction 9
FILMELEVL

SEQ ID NO: 10 - protein, 9 aa, artificial sequence
  Antigen epitope prediction 10
EPATKLPPL

SEQ ID NO: 11 - DNA, 48 bp, artificial sequence
  CDR3 DNA sequence
tgtgccagcagttcccgactagcgggggggacagatacgcagtatttt

SEQ ID NO: 12 - protein, 16 aa, artificial sequence
  CDR3 amino acid sequence
CASSSRLAGGTDTQYF

SEQ ID NO: 13 - DNA, 39 bp, artificial sequence
  CDR3 DNA sequence
tgtgccaccagcagggagggaggggagacccagtacttc

SEQ ID NO: 14 - protein, 13 aa, artificial sequence
  CDR3 amino acid sequence
CATSREGGETQYF

SEQ ID NO: 15 - DNA, 51 bp, artificial sequence
  CDR3 DNA sequence
tgtgccaccagcagagattggctatccaacgggaacactgaagctttcttt

SEQ ID NO: 16 - protein, 17 aa, artificial sequence
  CDR3 DNA sequence
CATSRDWLSNGNTEAFF

SEQ ID NO: 17 - DNA, 42 bp, artificial sequence
  CDR3 DNA sequence
tgtgccagcagttactcgctcggaatctacgagcagtacttc

SEQ ID NO: 18 - protein, 14 aa, artificial sequence
  CDR3 amino acid sequence
CASSYSLGIYEQYF

SEQ ID NO: 19 - DNA, 48 bp, artificial sequence
  CDR3 DNA sequence
tgtgccagcagtgatttcggaccatcgtggggcaatgagcagttcttc

SEQ ID NO: 20 - protein, 16 aa, artificial sequence
  CDR3 amino acid sequence
CASSDFGPSWGNEQFF

SEQ ID NO: 21 - DNA, 42 bp, artificial sequence
  CDR3 DNA sequence
tgtgccagcagcttagaggcagtgaacactgaagctttcttt

SEQ ID NO: 22 - protein, 14 aa, artificial sequence
  CDR3 amino acid sequence
CASSLEAVNTEAFF

SEQ ID NO: 23 - DNA, 42 bp, artificial sequence
  CDR3 DNA sequence
tgtgccagcagccccgttggcggccaaggaaccctccacttt

SEQ ID NO: 24 - protein, 14 aa, artificial sequence
  CDR3 amino acid sequence
CASSPVGGQGTLHF

SEQ ID NO: 25 - DNA, 39 bp, artificial sequence
  CDR3 DNA sequence
tgtgccagcagcttagggacactatatggctacaccttc

SEQ ID NO: 26 - protein, 13 aa, artificial sequence
  CDR3 amino acid sequence
CASSLGTLYGYTF

SEQ ID NO: 27 - DNA, 48 bp, artificial sequence
  CDR3 DNA sequence
tgcgccagcagtgagtctgggacagggttctcctacgagcagtacttc

SEQ ID NO: 28 - protein, 16 aa, artificial sequence
  CDR3 amino acid sequence
CASSESGTGFSYEQYF

SEQ ID NO: 29 - DNA, 42 bp, artificial sequence
  CDR3 DNA sequence
tgtgccagtagtgcggcaggggggccctacgagcagtacttc

SEQ ID NO: 30 - protein, 14 aa, artificial sequence
  CDR3 amino acid sequence
CASSAAGGPYEQYF

SEQ ID NO: 31 - DNA, 48 bp, artificial sequence
  CDR3 DNA sequence
tgtgccatcagtgagtcggacagtcccatcgacactgaagctttcttt

SEQ ID NO: 32 - protein, 16 aa, artificial sequence
  CDR3 amino acid sequence
CAISESDSPIDTEAFF

SEQ ID NO: 33 - DNA, 39 bp, artificial sequence
  CDR3 DNA sequence
tgcgccagcagcttggccgagggagatggctacaccttc

SEQ ID NO: 34 - protein, 13 aa, artificial sequence
  CDR3 amino acid sequence
CASSLAEGDGYTF

SEQ ID NO: 35 - DNA, 39 bp, artificial sequence
  CDR3 DNA sequence
tgtgccaccagcagagtggccggggagacccagtacttc

SEQ ID NO: 36 - protein, 13 aa, artificial sequence
  CDR3 amino acid sequence
CATSRVAGETQYF

SEQ ID NO: 37 - DNA, 39 bp, artificial sequence
  CDR3 DNA sequence
tgtgccaccagtgatggggcaggggtcgagcagttcttc

SEQ ID NO: 38 - protein, 13 aa, artificial sequence
  CDR3 amino acid sequence
CATSDGAGVEQFF

SEQ ID NO: 39 - DNA, 42 bp, artificial sequence
  CDR3 DNA sequence
tgtgccagcagtttggcggagatcaatcagccccagcatttt

SEQ ID NO: 40 - protein, 14 aa, artificial sequence
  CDR3 amino acid sequence
CASSLAEINQPQHF

SEQ ID NO: 41 - DNA, 39 bp, artificial sequence
  CDR3 DNA sequence
tgtgccagcagctcttcaagcgggaccggggctttcttt

SEQ ID NO: 42 - protein, 13 aa, artificial sequence
  CDR3 amino acid sequence
CASSSSSGTGAFF

SEQ ID NO: 43 - DNA, 39 bp, artificial sequence
  CDR3 DNA sequence
tgtgccagcagctcccttggggtcactgaagctttcttt

SEQ ID NO: 44 - protein, 13 aa, artificial sequence
  CDR3 amino acid sequence
CASSSLGVTEAFF

SEQ ID NO: 45 - DNA, 42 bp, artificial sequence
  CDR3 DNA sequence
tgtgccagcagcccaccgggcttgaatgaaaaactgtttttt

SEQ ID NO: 46 - protein, 14 aa, artificial sequence
  CDR3 amino acid sequence
CASSPPGLNEKLFF

SEQ ID NO: 47 - DNA, 48 bp, artificial sequence
  CDR3 DNA sequence
tgtgccagcagtgaagagggcagcccccggggatacgagcagtacttc

SEQ ID NO: 48 - protein, 16 aa, artificial sequence
  CDR3 amino acid sequence
CASSEEGSPRGYEQYF

SEQ ID NO: 49 - DNA, 42 bp, artificial sequence
  CDR3 DNA sequence
tgcgccagcagtcggacaggggagaattcacccctccacttt

SEQ ID NO: 50 - protein, 14 aa, artificial sequence
  CDR3 amino acid sequence
CASSRTGENSPLHF

SEQ ID NO: 51 - protein, 133 aa, artificial sequence
  Known TCR-a amino acid sequence
MMKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVNFGGGKLIFGQGTELSVKPN

SEQ ID NO: 52 - DNA, 399 bp, artificial sequence
  Known TCR-a base sequence
atgatgaaatccttgagagttttactagtgatcctgtggcttcagttgagctgggtttggagccaacagaaggaggtggagcagaattctggacccctcagtgttccagagggagccattgcctctctcaactgcacttacagtgaccgaggttcccagtccttcttctggtacagacaatattctgggaaaagccctgagttgataatgttcatatactccaatggtgacaaagaagatggaaggtttacagcacagctcaataaagccagccagtatgtttctctgctcatcagagactcccagcccagtgattcagccacctacctctgtgccgtgaacttcggaggaggaaagcttatcttcggacagggaacggagttatctgtgaaacccaat

SEQ ID NO: 53 - protein, 131 aa, artificial sequence
  Known TCR-b amino acid sequence
MRIRLLCCVAFSLLWAGPVIAGITQAPTSQILAAGRRMTLRCTQDMRHNAMYWYRQDLGLGLRLIHYSNTAGTTGKGEVPDGYSVSRANTDDFPLTLASAVPSQTSVYFCASSLSFGTEAFFGQGTRLTVV

SEQ ID NO: 54 - protein, 135 aa, artificial sequence
  substituted TCR-b amino acid sequence
MRIRLLCCVAFSLLWAGPVIAGITQAPTSQILAAGRRMTLRCTQDMRHNAMYWYRQDLGLGLRLIHYSNTAGTTGKGEVPDGYSVSRANTDDFPLTLASAVPSQTSVYFCATSRDWLSNGNTEAFFGQGTRLTVV